(19) United States
(12) Patent Application Publication
(10) Pub. No.: US 2010/0274266 A1
(43) Pub. Date: Oct. 28, 2010

(54) ROTARY TACK

(76) Inventors: Ofir Rimer, Kfar Truman (IL); Nir Altman, Kibbutz Kfar Etzion (IL); Shalom Levin, Atlit (IL); Asaf Levin, Atlit (IL); Izhak Fabian, Kfar Truman (IL); Aharon Cohen, Zichron Ya'akov (IL)

Correspondence Address: David Klein;DEKEL PATENT LTD., Beit HaRof'im, 18 Menuha VeNahala Street, Room 27, REHOVOT, IL

(21) Appl. No.: 12/427,780

(22) Filed: Apr. 22, 2009

(51) Int. Cl. A61B 17/08

(52) U.S. Cl. 606/151

(57) ABSTRACT

A rotary tack including a helical body constructed of a resorbable material and having a closed-loop base and a helix of spiral coils that extend from the base which terminate in a tip for piercing tissue, wherein each of the coils has a cross-section with an inner perimeter including an arcuate portion and a straight portion contiguous to each other.

### FIELD OF THE INVENTION

The present invention relates generally to rotary tacks, such as those used for hernia repairs and the like in laparoscopic and endoscopic procedures.

### BACKGROUND OF THE INVENTION

Tacks or other tissue fasteners made of resorbable or biodegradable materials (the terms being used interchangeably throughout) are well known. For example, U.S. Pat. Nos. 4,884,572 and 4,895,148 to Bays et al., the disclosures of which are incorporated herein by reference, describe repair tacks of biodegradable material chosen to have a degradation time in excess of the required healing time for the tissue.

### SUMMARY OF THE INVENTION

The present invention seeks to provide a rotary tack made of a resorbable material, as is described more in detail hereinbelow.

There is thus provided in accordance with an embodiment of the present invention a rotary tack including a helical body constructed of a resorbable material and having a closed-loop base and a helix of spiral coils that extend from the base which terminate in a tip for piercing tissue, wherein each of the coils has a cross-section with an inner perimeter including an arcuate portion and a straight portion contiguous to each other.

In accordance with one embodiment of the present invention the inner perimeter includes two straight portions that subtend an angle corresponding to two sides of a regular polygon and a single arcuate portion that extending from end points of the two straight portions. The regular polygon may be a pentagon.

In accordance with one embodiment of the present invention the arcuate portion and the straight portion are joined by a rounded corner. For example, the arcuate portion and the straight portions are joined by rounded corners and the two straight portions are joined by a rounded corner.

In accordance with one embodiment of the present invention an upper surface of the helix proximate the tip includes one or more steps.

In accordance with another embodiment of the present invention the arcuate portion includes arcuate spline grooves and the straight portion includes radial straight portions that connect one groove to another. For example, each of the arcuate spline grooves have a starting point and an end point, wherein the starting point starts at a smaller radius than the end point, and each of the radial straight portions connects the starting point of one groove to the end point of an adjacent groove.

### BRIEF DESCRIPTION OF THE DRAWINGS

The present invention will be understood and appreciated more fully from the following detailed description taken in conjunction with the drawings in which:

FIGS. 1A-1D are simplified pictorial illustrations of a rotary tack, constructed and operative in accordance with an embodiment of the present invention, wherein FIGS. 1C and 1D are respectively bottom and top views; and

FIGS. 2A-2C are simplified pictorial illustrations of a rotary tack, constructed and operative in accordance with another embodiment of the present invention, wherein FIGS. 2B and 2C are respectively bottom and top views.

### DETAILED DESCRIPTION OF EMBODIMENTS

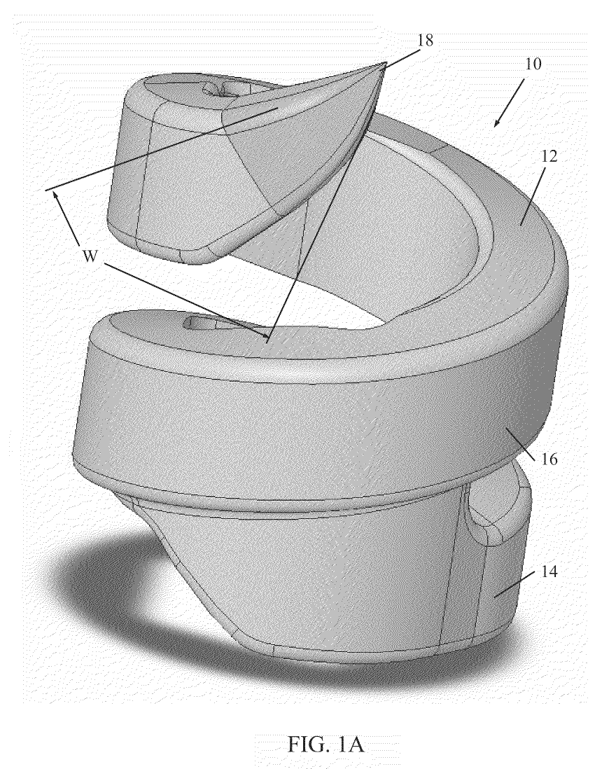

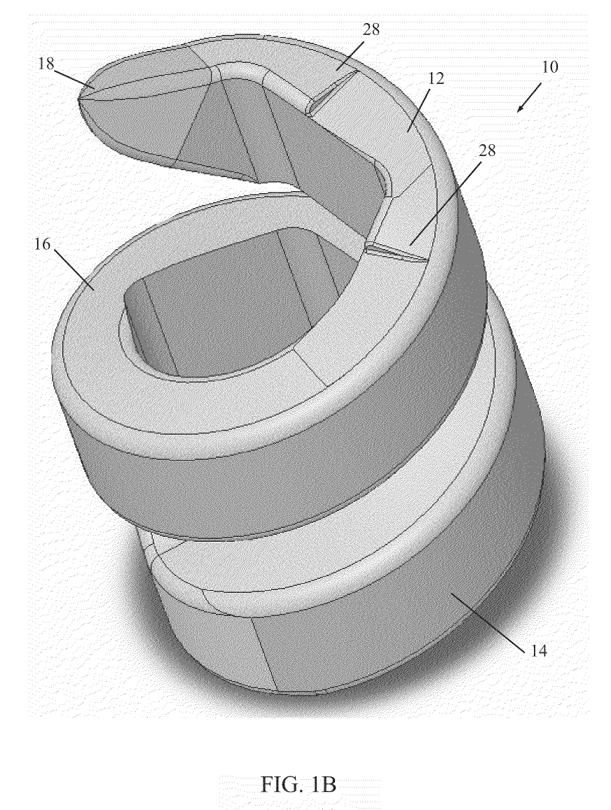

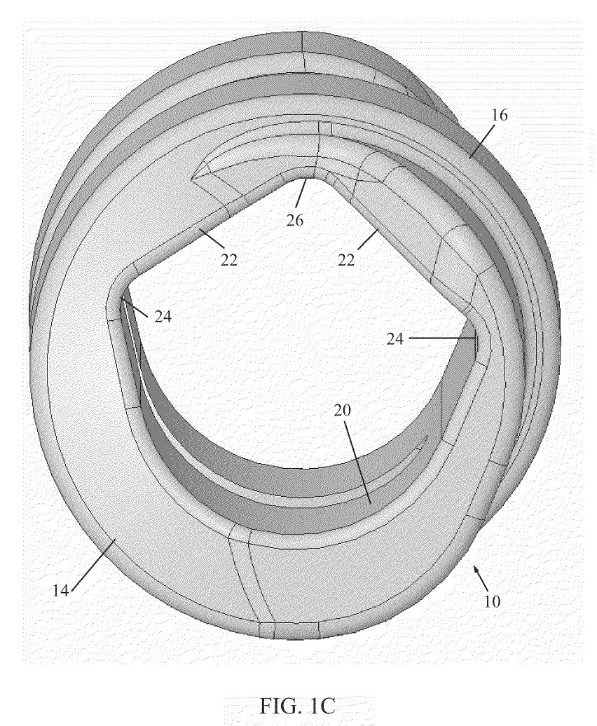

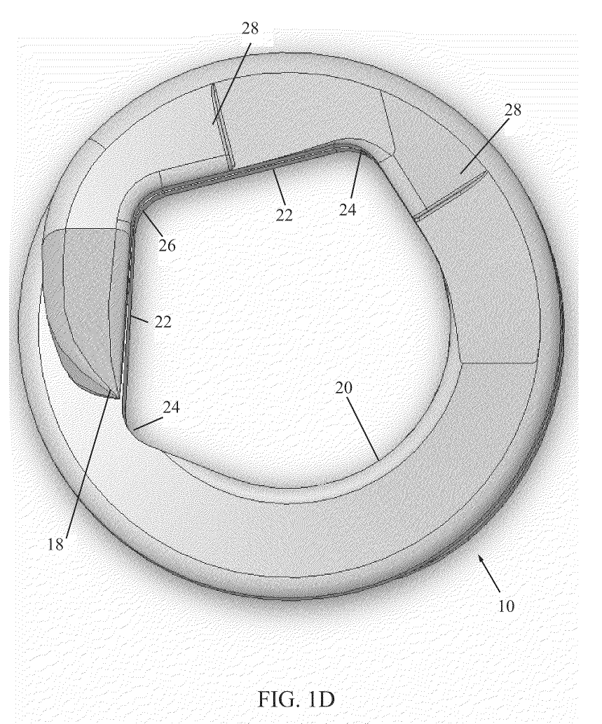

Reference is now made to FIGS. 1A-1D, which illustrate a rotary tack 10, constructed and operative in accordance with an embodiment of the present invention.

The tacks of the present invention are constructed of a resorbable material. The tacks may be constructed, without limitation, from a biodegradable polymer or copolymer of a type selected in accordance with the desired degradation time. That time, in turn, depends upon the anticipated healing time for the cartilaginous or other tissue which is the subject of the surgical procedure. Known biodegradable polymers and copolymers range in degradation time from about three months for polyglycolide to about forty-eight months for polyglutmic-co-leucine. A common biodegradable polymer used in absorbable sutures and the like is poly(L-lactide) which has a degradation time of about twelve to eighteen months. Without limitation, the tacks may be constructed from an absorbable copolymer derived from glycolic and lactic acids, such as a synthetic polyester chemically similar to other commercial available glycolide and lactide copolymers. Glycolide and lactide, in vivo, degrade and absorb by hydrolysis into lactic acid and glycolic acid which are then metabolized by the body.

In accordance with a non-limiting embodiment of the present invention, tack 10 includes a helical body 12 having a closed-loop base 14 and a helix of spiral coils 16 that extend from base 14 and which terminate in a tip 18 for piercing tissue (not shown). As seen in FIGS. 1C and 1D, each of the coils 16 has a cross-section with an inner perimeter including at least one arcuate portion 20 and at least one straight portion 22 contiguous to each other. In prior art helical fasteners, the base is open, that is, the lowermost coil is not a continuous closed coil or loop. In contrast, in the present invention, base 14 is closed. This geometry provides tack 10 with superior strength, especially important for use with the resorbable material.

In the embodiment of FIGS. 1A-1D, the inner perimeter includes two straight portions 22 that subtend an angle corresponding to two sides of a regular polygon and a single arcuate portion 20 that extending from end points of the two straight portions 22. The regular polygon may be a pentagon, wherein the subtended angle is 72°. The arcuate portion 20 and the straight portions 22 may be joined by a rounded corner. For example, the arcuate portion 20 and the straight portions 22 are joined by rounded corners 24 and the two straight portions are joined by a rounded corner 26. The inner perimeter comprises the drive section of tack 10, that is, the tack is placed on a complementary shaped drive shaft (not shown) which turns the tack in order to screw it into tissue (not shown).

As seen in FIG. 1A, the tip 18 has a sharp edge at a wide angle W aimed upwards relative to the helix. Angle W is much greater than the helix angle of the coils.

In accordance with one embodiment of the present invention an upper surface of the helix proximate tip 18 includes one or more steps 28 (this option not shown in FIG. 1A). Steps 28 may help prevent opening of the helix when pulling forces are applied to the tack 10.

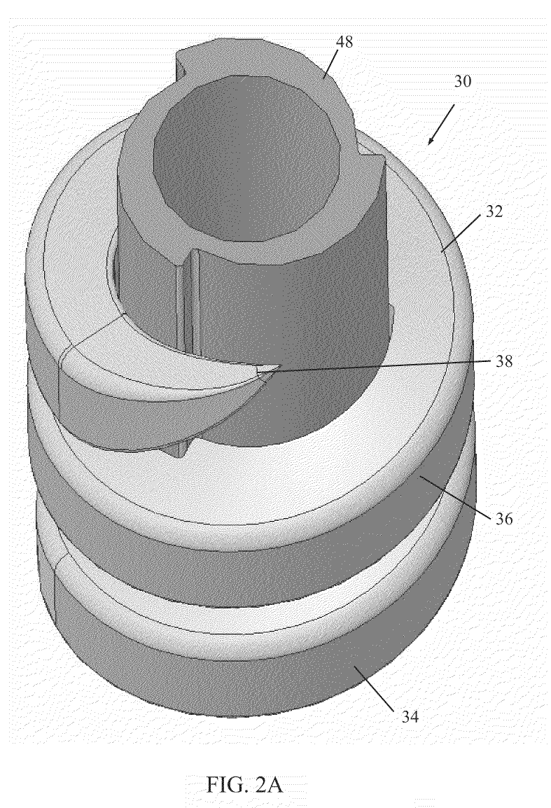

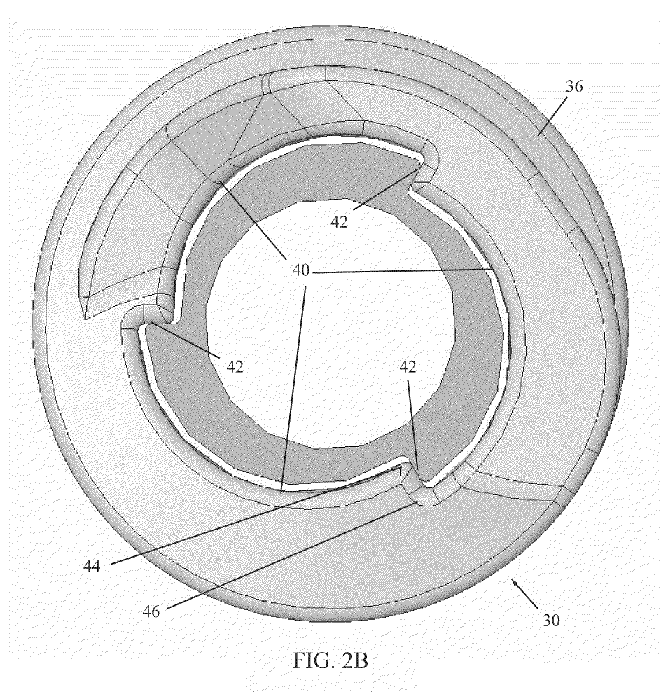

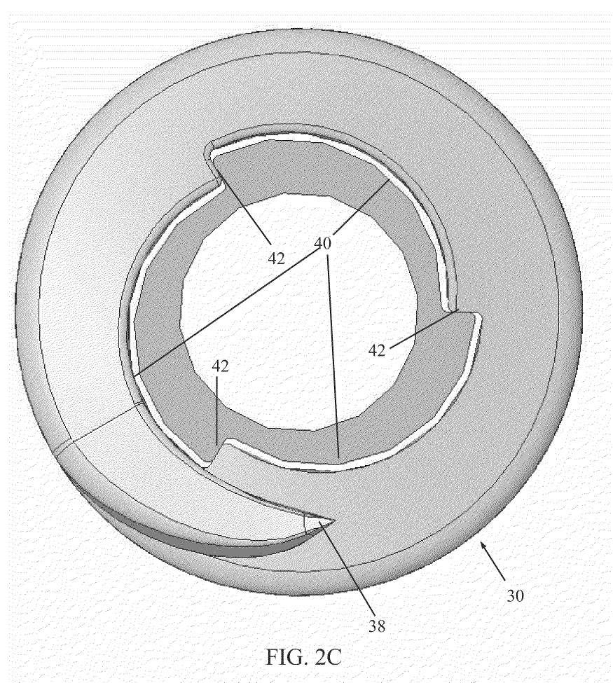

Reference is now made to FIGS. 2A-2C, which illustrate a rotary tack 30, constructed and operative in accordance with an embodiment of the present invention.

Tack 30 includes a helical body 32 having a closed-loop base 34 and a helix of spiral coils 36 that extend from base 34 and which terminate in a tip 38 for piercing tissue (not shown). As seen in FIGS. 2B and 2C, each of the coils 36 has a cross-section with an inner perimeter including at least one arcuate portion and at least one straight portion contiguous to each other.

In this embodiment the arcuate portion includes arcuate spline grooves 40 and the straight portion includes radial straight portions 42 that connect one groove 40 to another. For example, each of the arcuate spline grooves 40 have a starting point 44 and an end point 46, wherein the starting point 44 starts at a smaller radius than the end point 46. Each of the radial straight portions 42 connects the starting point 44 of one groove 40 to the end point 46 of an adjacent groove 40. Here too, the arcuate portions and the straight portions may be joined by rounded corners.

As before, the inner perimeter comprises the drive section of tack 30, that is, the tack is placed on a complementary shaped drive shaft 48 (FIG. 2A) which turns the tack in order to screw it into tissue (not shown). Tack 30 may also have the steps formed on the upper surface thereof.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A rotary tack comprising:
a helical body constructed of a resorbable material and having a closed-loop base and a helix of spiral coils that extend from said base which terminate in a tip for piercing tissue, wherein each of said coils has a cross-section with an inner perimeter comprising an arcuate portion and a straight portion contiguous to each other.

2. The rotary tack according to claim 1, wherein said inner perimeter comprises two straight portions that subtend an angle corresponding to two sides of a regular polygon and a single arcuate portion that extending from end points of the two straight portions.

3. The rotary tack according to claim 2, wherein said regular polygon is a pentagon.

4. The rotary tack according to claim 1, wherein said arcuate portion and said straight portion are joined by a rounded corner.

5. The rotary tack according to claim 2, wherein said arcuate portion and said straight portions are joined by rounded corners and the two straight portions are joined by a rounded corner.

6. The rotary tack according to claim 1, wherein an upper surface of said helix proximate said tip comprises one or more steps.

7. The rotary tack according to claim 1, wherein said arcuate portion comprises arcuate spline grooves and said straight portion comprises radial straight portions that connect one groove to another.

8. The rotary tack according to claim 7, wherein each of said arcuate spline grooves have a starting point and an end point, wherein the starting point starts at a smaller radius than the end point, and each of said radial straight portions connects the starting point of one groove to the end point of an adjacent groove.
